(19) United States
(12) Patent Application Publication
(10) Pub. No.: US 2024/0181024 A1
(43) Pub. Date: Jun. 6, 2024

(54) TREATING RESPIRATORY INFECTIONS

(71) Applicants: Leslie Roy BERRY, Burlington (CA); Anthony Kam Chuen CHAN, Ancaster (CA); William JACKSON, Hamilton (CA); Attilio DIFIORE, West Jordan, UT (US)

(72) Inventors: Leslie Roy BERRY, Burlington (CA); Anthony Kam Chuen CHAN, Ancaster (CA); William JACKSON, Hamilton (CA); Attilio DIFIORE, West Jordan, UT (US)

(73) Assignee: ATH Therapeutics Inc., Alexandria, VA (US)

(21) Appl. No.: 18/552,534

(22) PCT Filed: Mar. 25, 2022
(86) PCT No.: PCT/US2022/021859

§ 371 Date: Sep. 26, 2023

Related U.S. Application Data
(60) Provisional application No. 63/166,831, filed on Mar. 26, 2021.

(51) Int. Cl. A61K 38/55; A61K 9/00; A61K 47/61

(52) U.S. Cl. CPC A61K 38/55; A61K 9/0019; A61K 9/0078; A61K 47/61

(57) ABSTRACT

A method of treating or preventing a respiratory infection can include delivering aerosolized droplets of an antiviral solution to lung tissue of a subject, the antiviral solution including an aqueous carrier and an ATH conjugate that is solubilized in the aqueous carrier. The ATH conjugate includes antithrombin covalently bonded to heparin, the heparin including a polysaccharide chain having from about 15 to about 1,500 monosaccharide units.

### BACKGROUND

Due to the recent outbreak of COVID-19, there has been a lot of interest in vaccines and therapeutics for treating coronaviruses both prophylactically and to ameliorate the symptoms and risks associated with viral infections. There are also other viruses that can be dangerous to a percentage of people that become infected, sometimes resulting in death. The treatment of viruses in recent years has primarily been through wide scale vaccination programs. However, once infected, a vaccine is not useful. Furthermore, some people may have sensitivities or other reasons why vaccination is not practical, so the use of therapeutics is becoming more desirable for either prophylaxis or post-infection treatment.

Heparin is a medication that has been used primarily for treatment related to thrombosis. The mechanism by which heparin works is through ionically binding an antithrombin molecule in circulation and enhancing the ability of the antithrombin to neutralize coagulation factors that cause overt thrombosis clotting, which can lead to heart disease and stroke. However, the complex of antithrombin and heparin (or antithrombin heparin complexes) formed naturally in the body is relatively unstable allowing heparin and antithrombin to dissociate and re-associate. Thus, there are time frames where heparin molecules are free and unbound to antithrombin. Free heparin can in some instances cause serious bleeding and other unwanted side effects.

Heparin is very electronegative and could be useful for neutralizing surface proteins of viruses that are used to attach and infect native cells within a host. However, aside from free heparin being somewhat dangerous in some instances with respect to bleeding when preset at damaged tissue, heparin delivered directly to tissue, such as the lungs, for purposes of treatment against viruses is not particularly beneficial as it may not stay in the lungs for long enough for effective treatment, e.g., it may pass through the tissue and into systemic circulation in the case of the lungs. As many respiratory viruses such as COVID-19 can become concentrated in the respiratory systems, e.g., lung tissue, it would be desirable to provide a therapeutic compound to the respiratory systems of subjects, e.g., nasal canal and sinuses, epiglottis, larynx, trachea, bronchi, alveoli, or bronchiole, etc., to treat for viral infections with some longevity, and if there is tissue damage, in a way that the therapeutic compound would not exacerbate excess bleeding.

### BRIEF DESCRIPTION OF THE DRAWINGS

FIG. 1 is a schematic representation of an example antithrombin-heparin conjugate (ATH conjugate) electrostatically attracted to a virus in accordance with the present disclosure.

FIG. 2 is a schematic representation of an example nebulizer with an antiviral solution delivering antiviral solution droplets via inhalation to a subject in accordance with the present disclosure.

### DETAILED DESCRIPTION

The present disclosure is drawn to methods and systems (or kits) for preventing and/or treating respiratory and other infections. For example, this treatment can be used to neutralize viruses when a subject becomes exposed to the virus or has been infected with the virus, and/or treat the subject for viral disease resulting from viral infection. Furthermore, the present disclosure can in some instances also provide secondary benefits of treatment related to amelioration of thrombosis or clotting that may occur, in many cases without the risk of excess bleeding.

In one example, a method of treating or preventing a respiratory infection can include delivering aerosolized droplets of an antiviral solution to lung tissue of a subject. The antiviral solution can include an aqueous carrier and an antithrombin-heparin conjugate (or ATH conjugate) that is solubilized in the aqueous carrier. The ATH conjugate in this example includes antithrombin covalently bonded to heparin, and the heparin can include a polysaccharide chain having from about 15 to about 1,500 monosaccharide units.

In another example, a respiratory tissue treatment kit can include an antiviral solution and an aerosolizing device to form aerosolized droplets of the antiviral solution. The antiviral solution can include an aqueous carrier and an ATH conjugate that is solubilized in the aqueous carrier. The ATH conjugate can include antithrombin covalently bonded to heparin. The heparin in this example includes a polysaccharide chain having from about 15 to about 1,500 monosaccharide units.

In accordance with these examples, the aerosolized droplets can be delivered using any of a number of devices, such as a nebulizer, e.g., a jet nebulizer, an ultrasonic wave nebulizer, vibrating mesh nebulizer, etc.; an inhaler, e.g., pump inhaler, soft mist inhaler, powder delivery inhalator; sprayer, e.g., spray bottle; nasal delivery device, e.g., nasal irrigator; etc. If a nebulizer or other similar device is used, a single treatment dose may last, without limitation, from about 30 seconds to about 2 hours, from about 1 minute to about 2 hours, from about 2 minutes to about 1 hour, from about 3 minutes to about 2 hours, from about 5 minutes to about 45 minutes, from about 15 minutes to about 45 minutes, or from about 30 minutes to about 2 hours. In one example, the antiviral solution can be loaded or loadable in the aerosolizing device at a sufficient volume to deliver the aerosolized droplets for a period of time from about 1 minute to about 2 hours, or from about 5 minutes to about 2 hours. In another example, the sufficient volume delivered during the period of time provides delivery of about 0.05 mg to about 500 mg of the ATH conjugate to the alveoli of the lungs. If using an inhaler, the aerosolized droplets can be delivered in from about 1 to about 10 inhalation doses, from about 1 to about 5 inhalation doses, or from about 1 to about 3 inhalation doses, e.g., one dose may be a single pump or mechanical action used to eject the droplets. In some examples, the antiviral solution can be loaded or loadable in the aerosolizing device at a sufficient volume to deliver the aerosolized droplets using one or multiple single inhalation dosages to deliver about 0.05 mg to about 500 mg of the ATH conjugate to the alveoli of the lungs. By delivering in this manner, the ATH conjugate can remain at the lung tissue of the subject at detectable levels and does not substantially enter the bloodstream, e.g., no more than about 1% of ATH conjugate molecules, for a period of time of at least 1 day, at least 4 days, at least 7 days, etc. In one example, the ATH conjugate is present as a mixture of ATH conjugates in the antiviral solution. The mixture of ATH conjugates can have an average polysaccharide chain length from about 10 to about 1,200 monosaccharide units, from about 18 to about 1,000 monosaccharide units, or from about 50 to about 500 monosaccharide units. In another example, the antithrombin can be covalently bonded to the polysaccharide chain at a monosaccharide positioned at a first end of a polysaccharide chain. The ATH conjugate can include a polysaccharide chain having a chain length, a configuration relative to the antithrombin, or both that inhibits viral- or vaccine-induced thrombosis at the lung tissue In further detail, the polysaccharide chain may include a pentasaccharide sequence that interacts with the antithrombin. A first side of the pentasaccharide sequence can include a polysaccharide residue having a monosaccharide chain length from about 2 to about 15 and can further include a monosaccharide unit that is covalently bonded to the antithrombin. A second side of the pentasaccharide sequence can include a free polysaccharide chain having a monosaccharide chain length from about 10 to about 1,000 monosaccharides. In another example, the ATH conjugate can be present in the aqueous carrier at from about 0.005 wt % to about 2 wt %, e.g., partially solubilized or fully solubilized. In some examples, the ATH conjugate can be fully solubilized in the aqueous carrier and can be present in the aqueous carrier at from about 0.01 wt % to about 1 wt %. In some instances, depending on the aqueous carrier, some of the content over 1 wt % may or may not also be solubilized. In further detail, the aerosolized droplets can have an average droplet size from about 0.1 μm to about 10 μm based on droplet number (or the number of droplets) for some types of devices, e.g., nebulizer to deeper lung tissue. In other examples the aerosolized droplets can have an average droplet size from 1 μm to about 200 μm based on droplet number for other types of devices, e.g., sprayer or other upper respiratory irrigation device for more local delivery to nasal tissue, sinus tissue, or epiglottis tissue. The antiviral solution can be delivered to the subject as aerosolized droplets at volume from about 0.5 mL to about 1,000 mL. From about 0.1 mg to about 2 g, or from about 5 mg to about 200 mg, of the ATH conjugate can be delivered to the lung tissue during a single treatment, which can be a single pump, multiple pumps, or nebulized treatment over a period of time. Treatments can be repeated periodically with more or less ATH conjugate given as prescribed or directed by a medical professional, for example. Thus, treating the respiratory infection can be in the form of prophylaxis, e.g., preventing or reducing the viral load that may occur from infection, or in the form of treatment after infection, reducing viral load or eliminating the virus in the subject. Viral infections that may be treated (prophylactically or after infection) with this approach can include infections from a coronavirus, e.g., SARS-CoV-2 (COVID-19), SARS-COV-1, or a mutation or derivative thereof. Other viral infections that can be treated can be from an influenza virus, a respiratory syncytial virus (RSV), a parainfluenza virus, a human metapneumovirus (HMPV), a rhinovirus, an adenovirus, or a human bocavirus (HBoV).

In another example, a respiratory tissue treatment kit or device can include antiviral particles including ATH conjugate, wherein the ATH conjugate includes antithrombin covalently bonded to heparin, and wherein the heparin including a polysaccharide chain having from about 15 to about 1,500 monosaccharide units. This respiratory tissue treatment kit or device can also include a dry medicament inhaler loaded or loadable with the antiviral providing a metered dose or multiple metered dosages of the antiviral particles for inhalation. In one example, the antiviral (dry) particles can have an average particle size from about 0.1 μm to about 10 μm based on droplet number. In another example, the antiviral particles can be lyophilized particles of an antiviral solution containing the ATH conjugate.

In another example, a method of treating a viral infection can include delivering an antiviral composition to a subject (prophylaxis and/or treatment after viral infection). The antiviral composition can be in the form of an antiviral solution or antiviral particles, and can include an ATH conjugate including antithrombin covalently bonded to heparin. The heparin in this example includes a polysaccharide chain having from about 15 to about 1,500 monosaccharide units. The antiviral composition in the form of an antiviral solution may be delivered by any administration route, such as parenteral, intravenous, subcutaneous, intramuscular, oral, dermal, ocular, mucosal, or inhalation. In one specific example, the antiviral solution can include, for example, fully solubilized ATH conjugate in an aqueous carrier suitable for intravenous infusion or subcutaneous injection, and delivery is by intravenous infusion or subcutaneous injection. The antiviral solution, for example, can include the ATH conjugate in an aqueous carrier at from about 0.005 wt % to about 2 wt %. In one example, from about 0.1 mg to about 2 g of the ATH conjugate can be delivered to the subject during a single treatment. In another example, a single dose of the antiviral solution for delivery to the subject can be from about 0.5 mL to about 1,000 mL. In another example, the antiviral composition can be in the form of antiviral particles and can be delivered by any practical administration route, such as by oral, mucosal, or inhalation delivery. In further detail, the antiviral composition can be in the form of solid particles or powder, semi-solid particles, a solubilized liquid, a liquid dispersion, a compressed tablet, a suppository, a liquid-containing soft-gel capsule, a particle-containing capsule, or an emulsion. The mixture of ATH conjugates can have an average polysaccharide chain length from about 10 to about 1,200 monosaccharide units, or from about 50 to about 1,000 monosaccharide units. In another example, the antithrombin can be covalently bonded to the polysaccharide chain at a monosaccharide positioned at a first end of a polysaccharide chain. The polysaccharide chain may include a pentasaccharide sequence that interacts with the antithrombin. A first side of the pentasaccharide sequence can include a polysaccharide residue having a monosaccharide chain length from about 2 to about 15 and can also include a monosaccharide unit that is covalently bonded to the antithrombin A second side of the pentasaccharide sequence can include a free polysaccharide chain having a monosaccharide chain length from about 10 to about 1,000 monosaccharides. The ATH conjugate can be present in the aqueous carrier at from about 0.005 wt % to about 2 wt %, for example. From about 0.1 mg to about 2 g of the ATH conjugate can be delivered to systemic circulation during a single treatment. A single dose of the antiviral solution for delivery to the subject can be from about 0.5 mL to about 1,000 mL. In this example, the respiratory infection can be an infection from a coronavirus, e.g., SARS-COV-2 (COVID-19), SARS-COV-1, or a mutation or derivative thereof. For example, the electronegative moiety of the heparin can bind to a spike protein of the coronavirus. The viral infection can alternatively be an infection from an influenza virus, a respiratory syncytial virus (RSV), a parainfluenza virus, a human metapneumovirus (HMPV), a rhinovirus, an adenovirus, or a human bocavirus (HBoV).

It is noted that when discussing the methods of treating and/or preventing viral infections, and/or the respiratory tissue treatment kits, these discussions can be considered applicable to one another whether or not they are explicitly discussed in the context of that example. Thus, for example, when discussing an ATH conjugate related to the respiratory tissue treatment kits, such disclosure is also relevant to and directly supported in the context of the methods of treating and/or preventing viral infections, and vice versa.

Heparin is a medication that has been used for prophylaxis and treatment of thrombosis. The mechanism by which heparin works is through binding an antithrombin molecule in the circulation and enhancing the ability of the antithrombin to neutralize coagulation factors that cause overt thrombosis clotting, sometimes leading to heart disease and stroke. Binding between the heparin and the antithrombin may occur, for example, at a pentasaccharide sequence along the heparin molecule, but this binding may not be very permanent in many instances, and thus, the complex formed between antithrombin and heparin naturally, i.e. antithrombin heparin complexes with ionically bound pentasaccharide heparin unit with antithrombin, can be unstable so that heparin often dissociates from the antithrombin protein. This can result in unbound heparin molecules that can sometimes cause serious bleeding and other unwanted side effects if present at high enough concentrations.

In contrast and in accordance with the present technology, by covalently linking heparin to antithrombin, a more permanent and stable form of antithrombin-heparin is formed as a conjugate, referred to herein as “ATH conjugate” or “antithrombin-heparin conjugate.” ATH conjugate as prepared and used herein can provide enhanced clotting inhibition with reduced bleeding in many instances, making it a more desirable anticoagulant agent than heparin. To provide one example, the heparin chain that is covalently attached to the antithrombin may include a few monosaccharide residues immediately adjacent to the pentasaccharide sequence, e.g., from about 2 to about 15, from about 3 to about 10, or from about 3 to about 8 monosaccharides in a chain. One of those monosaccharide residues along that short chain of monosaccharides can be the point of covalent attachment to the antithrombin. In one example, the monosaccharide at the end of the saccharide residue chain can be the point of covalent attachment. With this covalent bond, there is a more permanent link between the heparin chain and the antithrombin protein, distinguishing the ATH conjugates of the present disclosure from less permanent, complexes of antithrombin and heparin where binding of antithrombin to the heparin pentasaccharide provides an example interaction of a less stable complex of antithrombin and heparin.

Due in part to the highly charged electronegativity of heparin or particularly long chain heparin, various viruses may strongly bind to heparin, thus neutralizing the virus, e.g., preventing the virus from infecting cells. In one example, virus that can be neutralized by heparin is SARS-COV-2, which is a coronavirus commonly referred to as COVID-19. By binding strongly to surface proteins of the coronavirus, e.g., the spike protein and/or other proteins at the surface, the spike protein can be blocked from binding with an intended host cell surface, e.g. at the ACE2 cell receptor, thus preventing infection. Without being bound by any specific numbers or mechanisms, it is believed that heparin has about a thousand times higher binding power to coronaviruses than that of the antibodies generated by the subject in response to receiving a vaccine. In particular, long heparin chains can be significantly better at viral spike protein binding than even shorter heparin chains. Once heparin has become attached and thus sterically blocks or even covers the spike proteins on the surface of the coronavirus, the coronavirus starts to disintegrate as the virus colony does not have a very long lifetime without its needed cellular infection lifecycle. Furthermore, the presence of heparin can have a secondary effect of inhibiting the deleterious effects of microthrombi formation, which is a clump of fibrin, platelets, and red blood cells induced by coronavirus infection. For example, fibrin can be deposited in the lung alveoli of a human subject, which can result in acute respiratory distress syndrome (ARDS) in seriously ill patients afflicted with a respiratory or pulmonary virus infection, such as a COVID-19 infection or other similar viral infection. In more specific detail, heparin functions by catalyzing the anticoagulant activity of the plasma protease inhibitor antithrombin. Unfractionated heparin (UFH) and its low molecular weight derivatives (LMWH) suffer from a number of shortcomings, including a short half-life, variable anticoagulant response, limited effectiveness at inhibiting thrombin (particularly clot-bound thrombin), induction of bleeding, and induction of thrombocytopenia. Furthermore, it is notable that the use of heparin for prophylaxis and/or treatment against viral infection in the airway can be challenging for a number of reasons. For example, antithrombin is not readily available in the airway tissues such as the nasal passage, pharynx, trachea, bronchus, or alveoli. Furthermore, with more specific reference to treatment of the lung tissue, heparin as a standalone molecule does not readily remain within the lungs with therapeutically effective longevity.

With respect to pulmonary delivery, ATH conjugate is not readily transported away from the lung tissue and into systemic circulation, and as a result, can remain there for a relatively long period of time at therapeutic levels until being naturally removed over time by the normal respiration process. For example, depending on how much is delivered to the lung tissue, ATH conjugate may remain at the lung tissue at therapeutically effective levels for greater than or equal to 12 hours, 24 hours, 48 hours, 3 days, 4 days, 5 days, 6 days, 1 week, or longer, e.g., from about 12 hours to about 2 weeks, from about 12 hours to about 10 days, from about 24 hours to about 1 week, from about 48 hours to about 1 week, from about 72 hours to about 2 weeks, from about 72 hours to about 10 days, or from about 72 hours to about 1 week. In some examples, for up to and even after 96 hours, there may be no detectable diffusion of ATH conjugate from the lungs into the blood. Because the ATH conjugate is not readily absorbed into the blood from the lungs, delivery of even a small amount of ATH conjugate to the lung airspace may provide relatively long term benefits for prophylaxis and/or treatment of many respiratory/pulmonary viruses, such as COVID-19, mutations or derivative thereof, other coronaviruses, and/or many other types of viruses that tend to concentrate and attack the lungs of an infected subject. Delivery of ATH conjugate of the present disclosure (with heparin covalently bonded to antithrombin) directly to the lung airspace can provide a therapeutic compound that binds with and neutralizes the virus itself, and can also have the secondary benefit of stopping pulmonary fibrin evolution from coagulation, thus inhibiting disease progression. For example, compared to traditional heparinoids, such as heparin, ATH conjugate has an increased half-life, decreased binding to plasma proteins and endothelial cells, and increased antithrombotic efficacy in animal models without increased risk of bleeding. In vitro, ATH conjugate directly inhibits several coagulation factors, with significantly increased rates compared to non-covalent antithrombin and unfractionated heparin mixtures (AT+H), and ATH conjugate can be more effective at inhibiting clot-bound thrombin compared to AT+H. Regarding binding, in some examples, because longer heparin chain lengths on average can be selected or engineered for covalent attachment during synthesis compared to that found in natural heparin, the highly negatively charged-based binding of the heparin chain (of the ATH conjugate) to the surface proteins of the virus can be very strong, e.g., from about 1.1 to about 8 times stronger than heparin, from about 2 to about 6 times stronger than heparin, or from about 3 to about 5 times stronger than heparin. In the specific case of coronaviruses, the significantly protruding spike proteins at the surface of the virus provide a good point of attraction with the highly electronegative heparin chain, and thus, may be at the higher end of these ranges, e.g., binding power from about 3 to about 8 times as strong as that of natural heparin. Furthermore, regarding the arresting of the pulmonary fibrin evolution related to coagulation, it is noted that even if there may be thrombosis due to leakage of coagulant plasma proteins into virus-infected and/or damaged lung tissue, this can be readily controlled by the long lasting effects of ATH conjugate within the airspace.

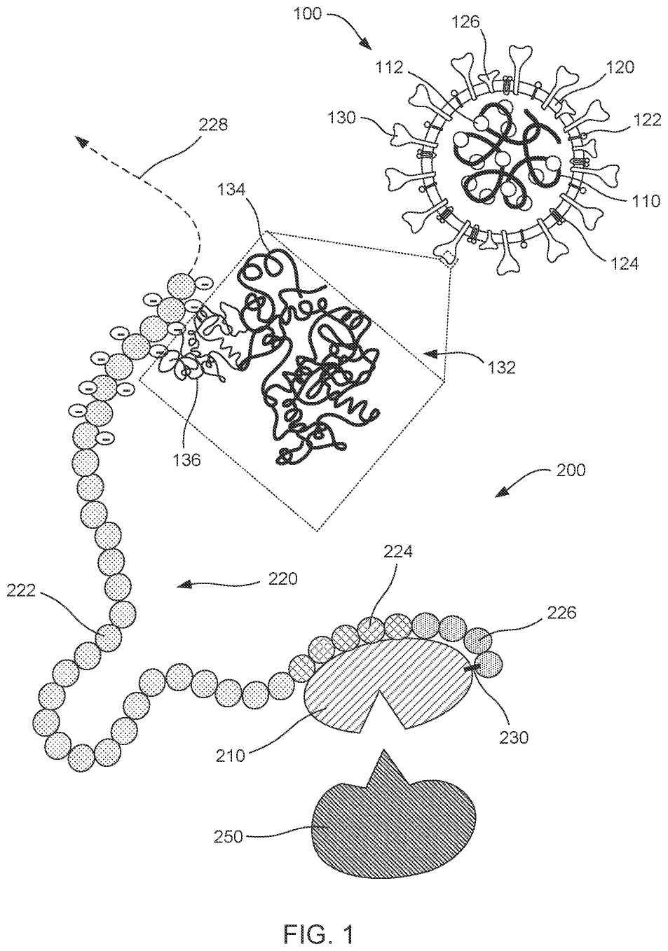

Referring now to FIG. 1, ATH conjugate 200 is shown including antithrombin 210 covalently bonded 230 to heparin 220 at a terminal or end-chain monosaccharide. The heparin, or heparin chain, includes a long chain of monosaccharides portion 222 (shown as being of variable lengths, including much longer monosaccharide lengths at 228). The heparin also includes a pentasaccharide sequence 224 and a few residue monosaccharides 226, which is the location of the covalent attachment to the antithrombin. Not all heparin chains include the pentasaccharide sequence, but this particular heparin chain is shown as also including the pentasaccharide sequence that naturally (but not permanently) binds to antithrombin. The pentasaccharide sequence can be used temporarily to attach to the antihrombin where conditions may be present to covalently attach the heparin (such as at a terminal monosaccharide) to the antithrombin. As can be seen, the heparin chain is highly electronegative, typically with several sulfonates, e.g., usually 1-3 per monosaccharide unit. The negative charges are shown by representation only, as they would be included along the entire chain length. Thrombin 250 is shown in this example for context, but is not part of the ATH conjugate.

In the example shown at FIG. 1, a virus 100, which in this example is a coronavirus, e.g., COVID-19, is shown. The coronavirus includes an RNA strand and nucleocapsid phosphoprotein carried by a lipid bilayer 120. The lipid bilayer carries various proteins, including an envelope glycoprotein 122, a membrane glycoprotein 124, a hemagglutinin-acetylesterase protein 126, and a spike glycoprotein, which is sometimes referred to as a “spike protein.” A more detailed expanded view of a small portion of the spike protein is shown at 132, and includes an angiotensin converting enzyme-2 receptor 134 and a receptor-binding domain 136. In this example, the heparin 220 of the ATH conjugate 200 is shown as being electrostatically attracted to the spike protein at the receptor-binding domain, though this electrostatic attraction could be elsewhere and may cover the virus more completely than shown in FIG. 1. This representation is for schematic purposes to show how the electronegative heparin of the ATH conjugate may interact with a surface of the virus to neutralize the virus.

Regarding the heparin chain length of ATH conjugate, in one example, the heparin molecule can have a monosaccharide chain length from about 15 to about 1,500, with an average chain length from about 10 to about 1,500, from about 15 to about 1,000, from about 20 to about 800, from about 50 to about 1,000, from about 50 to about 500, from about 100 to about 1,000, or from about 200 to about 400, for example. In further detail, there may be examples where less than about 10% of the ATH conjugate molecules have a heparin chain length of less than about 10 monosaccharide units, or less than about 5% of the ATH conjugate molecules have a heparin chain length of less than 10 monosaccharide units. In other examples, there may be embodiments where less than about 20% of the ATH conjugate molecules have a heparin chain length of less than about 25 monosaccharide units, or less than about 5% of the ATH conjugate molecules have a heparin chain length of less than 25 monosaccharide units. Notably, some of these shorter-length heparins that may be present on a plurality of the ATH conjugate molecules, e.g., with less than about 10 monosaccharide units, do not bind effectively with some viruses, such as coronaviruses. However, their presence, even if not effective for surface binding of the virus, may still benefit the composition as a whole by assisting with amelioration of thrombosis. In other examples, ATH conjugate can be administered to include from about 98% to 100% of the heparin chains having an average molecular weight from about 2,895 Daltons to about 434,250 Daltons, from about 5,211 Daltons to about 347,400 Daltons, from about 28,950 Daltons to about 217,125 Daltons, or from about 14,475 Daltons to about 144,750 Daltons. Antithrombin has an average molecular weight of about 58,200 Daltons, so the average molecular weight of the ATH conjugate molecule is the sum of the average molecular weights of the heparin polysaccharide and the antithrombin protein, i.e. 58,200 Daltons.

There are many routes of administration of solutions of ATH conjugate in accordance with the present disclosure. In one example, an ATH conjugate solution may be delivered intravenously or subcutaneously to treat the subject systemically for a viral infection. When treated intravenously or subcutaneously, for example, even very high doses of ATH conjugate can be given to a subject to approximate (or even exceed) maximum or near maximum viral inhibition because when delivered as the ATH conjugate molecule described herein (heparin covalently bonded to antithrombin), the conjugated heparin moiety has reduced activity that risks excess bleeding as a side effect.

On the other hand, particularly with viruses that attack the pulmonary systems, ATH conjugate may be particularly useful via delivery to the airways, e.g., via catheter or by delivery of liquid droplets. Though a catheter may be used to deliver fluid to the lungs, in some examples, it may be more efficacious to deliver ATH conjugate solution via a mist or an aerosolized fluid. In one example, a nasal mist of an ATH conjugate solution may be delivered to the nasal cavity and the pharynx, and to a lesser extent to the lungs, to provide prophylaxis or treatment for a respiratory virus. On the other hand and in further detail, by delivering a solution of ATH conjugate in the form of small droplets using a more specialized aerosolized dosing device, e.g., inhaler or nebulizer delivered via the mouth and/or nose, a larger portion of the ATH conjugate solution may be received effectively at the alveoli of the lungs, and thus, more surface area can be treated where there may be virus present, particularly if there are small enough droplets used to reach these deeper tissues of the lungs. Thus, the use of a nebulizer and/or an inhaler (or the like) where deeper areas of the lungs may receive droplets of ATH conjugate solution can enhance the effectiveness of treatment further in some instances. Still further, by using ATH conjugate instead of heparin alone, the chain length of the heparin found in ATH conjugate can in some instances be longer than that of heparin, thus providing an even stronger complexing power between the heparin moiety of the ATH conjugate and surface proteins of the virus.

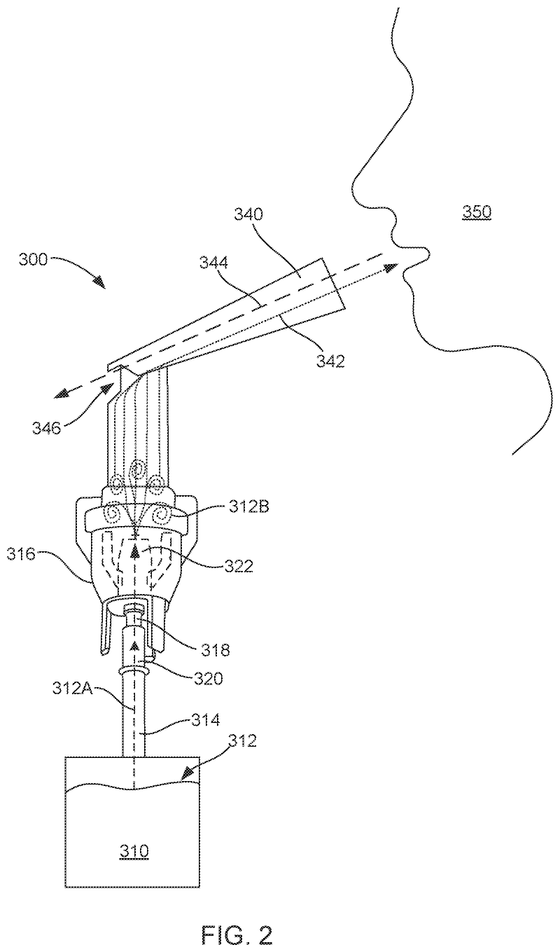

As an example, as shown in FIG. 2, a respiratory therapy device 300 is shown. In this example, the device is in the form of a pressurized nebulizer, but the nebulizer used to the droplets can be any of a number of devices, such as a jet nebulizer, an ultrasonic wave nebulizer, a vibrating mesh nebulizer, or the like. Alternatively, the respiratory therapy device could be in the form of an inhaler, such as a mechanical inhaler, e.g., pump inhaler, soft mist inhaler, etc. In this particular example, a container 310, which in this instance acts as a compressor, can hold the antiviral solution 312 of ATH conjugate where it can be pressurized to flow through a flow channel 314, e.g., flexible tubing or some other delivery channel, as a pressurized antiviral solution 312A. The flow channel can be coupled to a nebulizer cup 316 at a gas intake interface 318 via a coupler 320. The compressor and the flow channel may be considered peripheral equipment that is used with the nebulizer, or may be considered to be part of the nebulizer, depending on the arrangement. As the fluid is sent from the container to the nebulizer cup, upon fluidic interaction with a jetting architecture 322, the antiviral solution forms aerosolized droplets 312B of the antiviral solution to be inhaled 342 via a mouthpiece 340 by user 350. The user then exhales 344 and the airflow can escape out of an outlet opening 346.

In more detail, in some examples a jet nebulizer can be used that includes the pressurized antiviral solution which can be in the form of a fluid or a pressurized gas that is jetted through a nozzle through a thin layer of the antiviral solution to be converted to the aerosolized droplets. In some examples, the aerosolization can be assisted by the presence of one or more disruption member(s), such as a screen or a series of baffles, though other structures alone or in combination can be used to assist in the formation of the aerosolized droplets. In further detail, with this type of system, if the jet nebulizers are not 100% efficient (which may be typical), some of the aerosolized liquid medicament may not form an aerosol or multiple droplets may coalesce and fall to the bottom of the nebulizing cup where the antiviral solution may be recycled for further aerosolization. The recycling of the antiviral solution can avoid waste. If it is known that some nebulizers generate some amount or range of waste, then the total volume of antiviral fluid loaded in the nebulizer can be adjusted accordingly so that the subject receives the intended amount of ATH conjugate for the current treatment cycle by inhalation through the mouthpiece or mask.

With respect to ultrasonic wave nebulizers, this type of system can include a nebulizer cup loaded with the antiviral solution. In this example, a power source can be electrically coupled to a piezoelectric transducer which, when activated by the power source, generates vibration waves within the antiviral solution. The vibration waves cause a portion of liquid medicament at the surface to form aerosolized droplets. As with the jet nebulizer, aerosolized liquid medicament can be recycled by forming larger droplets that are returned to the antiviral solution reservoir. Furthermore, as with the jet nebulizer, the ultrasonic wave nebulizer can be configured to include disruption members in some examples, such as screens, baffles, etc. to increase the efficiency of the aerosolization process. Upon generating the aerosolized antiviral solution, the nebulizer can then deliver the droplets to the subject via inhalation using a mouthpiece or mask.

A vibrating mesh nebulizer can also be used and this type of device creates a mist through very small holes that vibrate at or near a liquid reservoir. As the mesh vibrates, the antiviral solution can become aerosolized.

In further detail regarding other types of respiratory therapy devices, as mentioned, any of a number of inhalers, or mechanically actuated inhalers, can be used in accordance with the present disclosure. For example, a pump inhaler can be used, which is similar to the single pump inhalers often used by those suffering from asthma. The spring-loaded pump for example, can be used to generate pressure so that a small metered dose of liquid can be ejected as an aerosol through a small nozzle(s). Likewise, a soft mist inhaler can be used to deliver a metered dose using manually rotated springs to build up tension around a flexible container and, when tension is released, pressure on the flexible container causes the liquid to be sprayed. This type of system does not use a gas propellant, such as pressurized air or oxygen, or a power source, as may be used with some more sophisticated nebulizer devices. In other examples, droplets may be generated using a nasal spray bottle, where the subject may insert a delivery nozzle in the nostril and squeeze or pump the nasal spray bottle to deliver droplets directly to the nasal cavity to be inhaled or to remain at or near the nasal cavity. Thus, delivery to the lung tissue may not be part of the treatment in some examples, as it may be sufficient in some instances to treat the nasal cavity and pharynx, for example, without deep lung inhalation that may occur when using a powered nebulizer or a more traditional mechanical inhaler. Thus, in accordance with examples of the present disclosure, a nasal spray bottle is considered to be an inhaler in accordance with examples of the present disclosure, as the antiviral solution carried therein can be used to form droplets that can be inhaled via the nasal passageway.

Though delivery dosages described in this example relate to directly treating the lung tissue with antiviral solutions of ATH conjugate via aerosolized droplets, there are other ways to directly treat the lungs, such as by the use of a catheter or intratracheal instillation. Those techniques can also be effective for treating the lung tissue with antiviral solution containing ATH conjugate. In other examples, ATH conjugate can be freeze-dried or lyophilized as a fine powder, and the powder can be delivered as a metered dose to the lungs using a medicament inhalator, for example.

If treating the subject by other routes other than by direct treatment of the air passages (or airways), such as for systemic circulation or localized delivery (other than by inhalation), administration can be via any accepted systemic or local route, for example, via parenteral, intravenous, subcutaneous, intramuscular, oral, dermal (e.g., transdermal or topical) ocular, or mucosal (e.g., transmucosal such as nasal, intraoral, anal, vaginal, etc.). The dosage may be in the form of solid, semi-solid or liquid dosage forms, such as for example, tablets, suppositories, pills, capsules, powders, solutions, suspensions, emulsions or the like, such as in unit dosage forms suitable for simple administration of precise dosages. Regardless of how it is administered, ATH conjugate can be formulated in a non-toxic, inert, pharmaceutically acceptable carrier medium, at a pH of about 3-8 or at a pH of about 6-8. Generally, the aqueous formulation can be compatible with the culture or perfusion medium. The compositions will include a conventional pharmaceutical carrier or excipient, and/or may include other medicinal agents, pharmaceutical agents, carriers, adjuvants, etc. The aqueous carrier may be in the form of saline in some examples In addition to water, which is typically a major carrier in antiviral solutions, additional aqueous carrier components can include various oils, including those of petroleum, animal, vegetable or synthetic origin, for example, peanut oil, soybean oil, mineral oil, sesame oil, and the like. Dextrose, mannitol, glycols, or the like are examples of suitable liquid carriers, particularly for injectable solutions. Suitable pharmaceutical carriers include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. In further detail, the antiviral solution (as a liquid or dried as particles, for example), may also contain minor amounts of non-toxic auxiliary substances such as organic co-solvent(s), surfactant(s), wetting or emulsifying agents, pH buffering agents such as sodium acetate, sorbitan monolaurate, triethanolamine oleate, etc., or the like. This list is not intended to be exhaustive, but only representative of how the antiviral solution of ATH conjugate may be formulated for delivery, either as aerosolized droplets to the airway, e.g., lungs, or otherwise.

Antithrombin-heparin conjugate (ATH conjugate) can be prepared by covalent attachment of heparin chains to antithrombin (AT). Heparin contains aldose termini which coexist in an equilibrium between hemiacetal and aldehyde forms. Heparin can be conjugated to antithrombin by Amadori rearrangement of the single Schiff base formed spontaneously between the aldose terminus aldehyde on heparin and a lysyl or N-terminal amino on the antithrombin or by reduction of the single Schiff base formed spontaneously between the aldose terminus aldehyde on heparin and a lysyl or N-terminal amino on the antithrombin The heparin is unmodified (unreduced in activities) prior to conjugation and is linked at one specific site at one end of the molecule without any unblocked activation groups or crosslinking of the antithrombin.

The reaction is typically carried out at a pH of about 4.5 to about 9, or at about 5 to about 8, or even at about 7 to about 8. The reaction is generally done in aqueous media. However, organic media, especially polar hydrophilic organic solvents such as alcohols, ethers and formamides and the like may be employed in proportions of up to about 40% to increase solubility or reactivity of the reactants, if necessary. Non-nucleophilic buffers such as phosphate, acetate, bicarbonate and the like may also be employed.

Imines formed by condensation of the amines of the antithrombin with the terminal aldose residues of the heparin can be reduced to the corresponding amines. This reduction may be accomplished concurrently with imine formation or subsequently. A wide array of reducing agents may be used, with hydride reducing agents, such as for example, sodium borohydride or sodium cyanoborohydride being specific examples that are useful. Generally, any reducing agent that does not reduce disulfide bonds can be used

Alternatively, if reduction of the intermediate imine is not desired, the imine may be incubated for a sufficient period of time, typically about 1 day to 1 month, more typically about 3 days to 2 weeks, to allow Amadori rearrangement of the intermediate imine. The terminal aldose residues of the heparin conjugated by the methods provided by this invention can possess C2 hydroxy groups on the terminal aldose residue, i.e., a 2-hydroxy carbonyl moiety which is converted to a 2-hydroxy imine by condensation with the amine of the antithrombin being conjugated to the heparin. In the Amadori rearrangement, the α-hydroxy imine (imine at C1, hydroxy at C2) formed by the initial condensation may rearrange to form an α-keto amine by enolization and re-protonation (keto at C2, amine at C1). The resulting α-carbonyl amine is thermodynamically favored over the precursor α-hydroxy imine, thus providing a stable adduct with minimal disruption of the heparin chain. Thus, heparin can be covalently conjugated at the C1 of the terminal aldose residue of the heparin to an amine-containing antithrombin chain via an amine linkage. Covalent conjugates of antithrombin and heparin can be formed by simply mixing heparin and antithrombin in a buffer and allowing a keto-amine to spontaneously form by an Amadori rearrangement between the heparin aldose terminus and an antithrombin lysyl or N-terminal amino group. Thus, the Amadori rearrangement can be used to prepare conjugates of heparin to AT. This is a particularly mild and simple method of conjugation, which minimizes the modification of the glycosaminoglycan, thus maximizing the retention of its biological activity.

Typically, heparin molecules with longer chain lengths can be more effective at inhibiting thrombin than heparin with lower chain lengths. On the other hand, heparin molecules with shorter chain length heparin may be effective at binding surface proteins of some viruses, such as the spike protein at the surface of a coronavirus. However, when heparin is part of an ATH conjugate, the heparin portion may be effective at both ameliorating thrombosis and binding the surface proteins of some viruses. In accordance with the present disclosure, typically the longer the heparin chain, a good balance between its affinity for surface proteins of many viruses and ameliorating thrombosis can be realized, e.g., a minimum chain length of at least 15, at least 18, at least 30, at least 50, at least 100, or at least 200 monosaccharide units. Thus, in one example, a heparin chain of at least about 18 monosaccharide units or more, (individually or on average) can be used effectively to bind both antithrombin and thrombin, and can also effectively neutralize a target virus being treated within a subject. Even longer chains can be beneficial for both purposes as well. More specifically, an average length of about 15 to about 1,500 monosaccharide units, from about 18 to about 1,500 monosaccharide units, from about 30 to about 1,500 monosaccharide units, from about 50 to about 1,500 monosaccharide units, from about 18 to about 1,000 monosaccharide units, from about 50 to about 1,000 monosaccharide units, from about 50 to about 500 monosaccharide units, from about 100 to about 1,500 monosaccharide units, from about 100 to about 1,000 monosaccharide units, from about 200 to about 1,000 monosaccharide units, or from about 200 to about 500 monosaccharide units can be present, for example. As there are many different monosaccharide units that may be present along a heparin chain, the molecular weight of an individual heparin molecule can vary to some degree. Likewise, the weight average molecular weight of a heparin-containing composition can likewise vary. As a reference, using a heparin molecule structure where there is a monosulfated uronic acid-disulfated glucosamine heparin disaccharide that is without the sodium or other ions found in a salt form, an 18 saccharide (9 disaccharide) chain may be around 5,200 Daltons.

Other processes to prepare the ATH conjugates of the present disclosure can likewise be considered for enhancing the effectiveness of the compound against viral or other infections. For example, ATH conjugates can, in some cases, be prepared using unfractionated heparin based on the number average of monosaccharide units described above. In other cases, however, ATH conjugate can be prepared using heparin from which low molecular weight heparin chains have been removed so that 98% or more of the ATH conjugate formed does not include short or fractionated heparin. Heparin is readily available in an unfractionated form, which contains molecules with a wide range of molecular weights. By removing from most to all of the heparin molecules having molecular weights less than 3,000 Daltons prior to conjugating the heparin with the antithrombin, the activity of the ATH conjugate can be enhanced. In an additional embodiment, heparin molecules having a molecular weight less than 5,000 Daltons can be from mostly to completely removed prior to conjugation with antithrombin. In another additional example, heparin molecules having a molecular weight less than 7,500 Daltons can be from mostly to completely removed prior to conjugation with antithrombin. In some examples, from 98% to 100% of the heparin molecules used to make the ATH conjugate can have a molecular weight for 3,000 Daltons or greater, 5,000 Daltons or greater, or 7,500 Daltons or greater. The amount of heparin that becomes conjugated with the antithrombin can be greater than 50%, greater than 70%, or greater than about 85% based on the total number of heparin molecules, for example. Likewise, the amount of antithrombin that becomes conjugated with the heparin can be greater than 50%, greater than 70%, or greater than about 85% based on the total number of antithrombin molecules, for example.

The ATH conjugates formed using heparin from which low molecular weight heparin molecules have been removed are compositionally different from ATH complexes (naturally occurring ionic complexes) or even other ATH conjugates that include a normal concentration of unfractionated heparin as is typically commercially available. In some examples, low molecular weight heparin chains, e.g., less than about 3,000 Daltons, less than about 5,000 Daltons, or less than about 7,500 Daltons, can be removed from the heparin prior to reaction with antithrombin to synthesize the ATH conjugate. Therefore, the ATH conjugate is devoid of low molecular weight heparin chains conjugated to the AT. Without being bound to any particular theory, it is believed that low molecular weight heparin chains (such as less than 3,000 Daltons, less than 5,000 Daltons, or less than 7,500 Daltons) compete with longer chain heparins for conjugating to AT. The very low molecular weight heparin chains have a high proportion of aldose termini which react with the AT. Therefore, the very low molecular weight heparin chains tend to conjugate with antithrombin more quickly, out-competing the higher molecular weight heparin chains. However, once the very low molecular weight heparin chains are bonded to the AT, the chains do not contain sufficient sites or length for binding thrombin and Factor Xa, an enzyme involved in the coagulation cascade. The inhibitory activity against factor Xa and thrombin drops dramatically in the lowest molecular weight range of heparin molecules. Thus, antithrombin heparin complexes and even ATH conjugates with higher concentrations of low molecular weight heparin may have very low (or even zero activity in some instances) for preventing thrombogenesis. Although commercial heparin contains a relatively small percentage of heparin chains below 5,000 Daltons, this concentration is still typically considerably higher than 2% based on molecule count. These very low molecular weight heparin chains have such a high reactivity with antithrombin so that a significant amount of the ATH conjugate formed contains the very low molecular weight heparin chains.

If the very low molecular weight heparin is not removed first, prior to conjugation with antithrombin, then a greater proportion of reactive termini in this population versus that of the higher molecular weight heparin will tend to outcompete the other heparin molecules to a varying degree across the entire molecular weight spectrum (as the proportion of aldose termini varies continually across the whole molecular weight range of heparin). This can have adverse effects on the final ATH conjugate. First, the ATH conjugate will contain a significant population of ATH conjugate molecules containing very small heparin chains with no activity. Second, the remaining ATH conjugate molecules (outside of this very low molecular weight range of ATH conjugate) will contain a population of heparin that has a reduced proportion of heparin chains in discrete molecular weight ranges that had fewer aldose termini to compete with the inactive low molecular weight heparin chains. This low aldose type heparin tends to be in the much longer chains but is not entirely defined by a straight relationship between heparin chain length and aldose termini required for linkage to AT.

The mechanism by which heparin binds antithrombin and thrombin is referred to as the template or bridging mechanism. Heparin can exert its effect via conformational activation by binding to antithrombin as an ionic complex at a specific pentasaccharide normally present on heparin molecules, and can allosterically convert the antithrombin into a structural form that is much more reactive towards coagulation proteases. Alternatively, heparin may act as a template through binding to both inhibitor and enzyme, thus localizing the molecules for reaction. In this mechanism, conformational activation of antithrombin by heparin occurs but additional reaction rate enhancement is gained by simultaneous binding of heparin to the enzyme, thus assisting approach of the coagulation factor towards the activated inhibitor.

Somewhat lower molecular weight heparin chains may be useful for inhibiting Factor Xa. A particular pentasaccharide sequence in heparin can bind to antithrombin and activate the antithrombin for inhibiting Factor Xa. The particular pentasaccharide sequence has been made available on its own as the pharmaceutical “Fondaparinux,” but the sequence can occur in heparin chains as well. The sequence of monosaccharides is shown in Formula I:

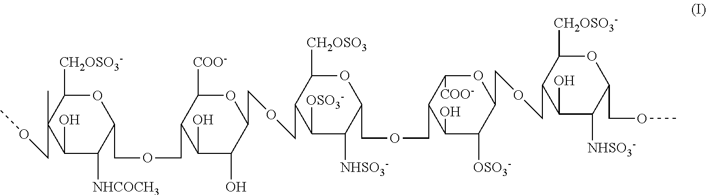

Another structure is shown in Formula II that is not drawn to the pentasaccharide sequence, but rather to a more general structure making up usually a significant portion of the heparin chain, e.g., about 60-95% or so of the heparin in various mammal structures or tissue. This common disaccharide along the heparin polysaccharide chain includes L-Iduronic acid and D-glucosamine, and is shown as follows:

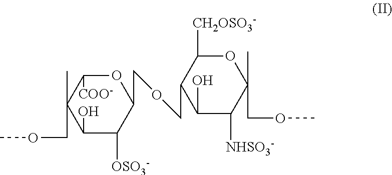

Variations of these individual monosaccharide units can also be present. For example, the disaccharides along the chain may average from about 2-3 sulfonate groups, though closer to 3 can be more common. There can also be other variants of monosaccharides or disaccharides along the heparin chain.

Heparin chains with less than 18 monosaccharides that contain this pentasaccharide sequence may be able to activate antithrombin to inhibit Factor Xa even though the chains are not long enough to bind to antithrombin and thrombin. On the other hand, longer heparin chains can in some cases have the highest inhibitory activity with respect to thrombosis. However, some mid-range and lower molecular weight heparin chains can have less undesirable binding to other plasma proteins and platelets. Therefore, these mid-range heparin chains can be more selective for inhibiting thrombin and factor Xa without causing unwanted side effects such as platelet dysfunction from binding with platelets and binding other materials. However, in accordance with the present disclosure and as shown in further detail elsewhere herein, when neutralizing a virus from a viral infection, such as at the lung tissue, longer chains can have more electronegative charge, and thus, can be even more effective in rendering the virus ineffective for further infection, thus causing the virus to die.

Isolating the higher molecular weight ATH conjugate after the conjugation to give very long chain ATH conjugate provides a less desirable and distinct product compared to the present technology which separates out (substantially or fully) the heparin prior to conjugation. For example, the proportion of 2-pentasaccharide high activity molecules in this subpopulation may be altered because of a differential ability of these high activity chains to compete with the very low molecular weight heparins for conjugation. Additionally, isolating the high molecular weight ATH conjugate after conjugation eliminates ATH conjugate molecules with mid-range and lower sized heparin chains that are also active and have other desirable characteristics such as reduced non-selective binding to plasma proteins and platelets.

Alternatively, attempts to react all aldose-terminating heparin chains with antithrombin by increasing the ratio of antithrombin to heparin in the reaction mixture are not likely to succeed because many experiments have shown that only up to 60 wt % conversion of antithrombin into ATH conjugate is obtained even with the aldose containing heparin in several-fold excess and at highest practical concentrations. Reducing the proportion of heparin to antithrombin even more will only decrease the ATH conjugate yield further without any promise that all of the active longer chains will be incorporated into the product.

In some more specific examples, a composition for preventing thrombogenesis can contain ATH conjugate formed from commercial heparin, where the heparin chains with a molecular weight less than 3,000 Daltons have been substantially removed or fully removed. In other embodiments, heparin chains with a molecular weight less than 5,000 Daltons can be substantially removed or removed. In other embodiments, heparin chains with a molecular weight less than 7,500 Daltons can be substantially removed or removed. By “substantially removed,” this refers to compositions with at least 98% of the heparin chains by heparin molecule count used to form the ATH conjugate have been removed, so that a maximum of only about 2% of the heparin that forms the ATH conjugate is below the target molecular weight, e.g., 3,000 Daltons, 5,000 Daltons, or 7,500 Daltons. Thus, in this example, the ATH conjugate product can contain heparin with 98% or greater of heparin chains that range in molecular weight from 3,000 Daltons (or 5,000 Daltons) up to the highest molecular weights contained in the commercial heparin or otherwise practical or possible. In certain examples, this range of molecular weights can be from 3,000 Daltons to 100,000 Daltons, from 3,000 Daltons to 50,000 Daltons, from 5,000 Daltons to 100,000 Daltons, from 5,000 Daltons to 50,000 Daltons, from 7,500 Daltons to 100,000 Daltons, or from 7,500 Daltons to 50,000 Daltons. In additional examples, at least a portion of the heparin chains can be in a mid-molecular weight range. For example, at least a portion of the 98% or more of heparin chains in the ATH conjugate can have a molecular weight from 3,000 Daltons to 30,000 Daltons, from 3,000 Daltons to 20,000 Daltons, from 3,000 Daltons to 15,000 Daltons, from 3,000 Daltons to 10,000 Daltons, from 5,000 Daltons to 30,000 Daltons, from 5,000 Daltons to 20,000 Daltons, from 5,000 Daltons to 15,000 Daltons, or from 5,000 Daltons to 10,000 Daltons. Thus, the ATH conjugate can be substantially devoid or devoid of heparin chains with a molecular weight below 3,000 Daltons or 5,000 Daltons.

Commercial heparin can typically contain a range of heparin chains with molecular weights ranging from 1,000 Daltons or less to 50,000 Daltons or more. The lowest molecular weight fraction, such as the chains with molecular weights below 3,000 or 5,000 Daltons, can be removed by any suitable method. Non-limiting examples of methods for removing the low molecular weight chains include dialysis, diafiltration, gel filtration and electrophoresis. Dialysis or diafiltration can be performed under high salt conditions. For example, high salt conditions for dialysis or diafiltration can include salt concentrations from about 1 M NaCl to about 4 M NaCl. Salts other than NaCl can also be used. The high salt concentration can assist movement of the small chains through membranes having appropriate pore sizes. Gel filtration can be performed using a suitable media for separating molecules by size. In one particular example, gel filtration can be performed on Sephadex® G-200, which is a gel media for separating molecules with molecular weights in the range of 1,000 to 200,000 Daltons. Commercial heparin can be gel filtered on a column of gel media, and a series of fractions can be eluted with the first fractions containing the highest molecular weight chains and the subsequent fractions containing progressively lower molecular weights. The molecular weights of heparin in each fraction can be determined, and the fractions having the desired molecular weights can be pooled. Using this method, fractions containing heparin with molecular weights below the threshold of 3,000 or 5,000 Daltons can be excluded. If desired, heparin chains above a certain threshold can also be excluded. For example, fractions containing heparin above 50,000 Daltons, 30,000 Daltons, 20,000 Daltons, 15,000 Daltons, or 10,000 Daltons can be excluded if desired. The pooled fractions having the desired range of molecular weights can then be used to synthesize ATH conjugate.

It should be noted that the methods of removing the very low molecular weight heparin chains described above are only exemplary and should not be considered limiting. Any method of processing commercial heparin to remove heparin chains below a certain threshold molecular weight can be used in the present disclosure.

In various embodiments of the present disclosure, the treatments and methods described herein can be performed using ATH conjugate having low molecular weight heparin removed, or alternatively, using ATH conjugate formed from unfractionated heparin.

ATH conjugate can be formed by conjugating antithrombin with the heparin that is now devoid of very low molecular weight chains. Exemplary methods of conjugating heparin with antithrombin are disclosed in U.S. Pat. No. 7,045,585, which is incorporated herein by reference. These methods can be applied to forming ATH conjugate using heparin from which the very low molecular weight chains have been removed, as described herein. Heparin can be conjugated with antithrombin through a simple one-step process, which provides for direct covalent attachment of the amine of an amine containing moiety (such as, but not limited to, amine containing oligo(poly)saccharides, amine containing lipids, proteins, nucleic acids and any amine containing xenobiotics) to a terminal aldose residue of a heparin chain. For forming ATH conjugate, the amine containing moiety is present in the AT, although other proteins can be conjugated using the same methods. The mild non-destructive methods provided herein allow for maximal retention of biological activity of the protein and allow direct linkage of the protein without the need for intermediate spacer groups.

In one embodiment, heparin is incubated with antithrombin at a pH suitable for imine formation between the amine and the terminal aldose or ketose residue of the heparin. Terminal aldose and ketose residues generally exist in an equilibrium between the ring closed cyclic hemiacetal or hemiketal form and the corresponding ring opened aldehyde or ketone equivalents. Generally, amines are capable of reacting with the ring opened form to produce an imine (Schiff base). Typically, the aldoses are more reactive because the corresponding aldehydes of the ring open form are more reactive towards amines. Therefore, covalent conjugate formation between amines and terminal aldose residues of heparin provides a method of attaching the antithrombin containing an amine to the heparin.

The reaction is typically carried out at a pH of about 4.5 to about 9, and more typically at about 5 to about 8, and even more typically at about 7 to about 8. The reaction is generally done in aqueous media. However, organic media, especially polar hydrophilic organic solvents such as alcohols, ethers and formamides and the like may be employed in proportions of up to about 40% to increase solubility or reactivity of the reactants, if necessary. Non-nucleophilic buffers such as phosphate, acetate, bicarbonate and the like may also be employed.

In some cases the imines formed by condensation of the amines of the antithrombin with the terminal aldose residues of the heparin are reduced to the corresponding amines. This reduction may be accomplished concurrently with imine formation or subsequently. A wide array of reducing agents may be used, such as hydride reducing agents including sodium borohydride or sodium cyanoborohydride. In one example, any reducing agent that does not reduce disulfide bonds can be used.

Alternatively, if reduction of the intermediate imine is not desired, the imine may be incubated for a sufficient period of time, typically about 1 day to 1 month, more typically about 3 days to 2 weeks, to allow Amadori rearrangement of the intermediate imine. The terminal aldose residues of the heparins conjugated by the methods provided by this disclosure frequently possess C2 hydroxy groups on the terminal aldose residue, i.e., a 2-hydroxy carbonyl moiety which is converted to a 2-hydroxy imine by condensation with the amine of the antithrombin being conjugated to the heparin. In the Amadori rearrangement, which is particularly common in carbohydrates, the α-hydroxy imine (imine at C1, hydroxy at C2) formed by the initial condensation may rearrange to form an α-keto amine by enolization and re-protonation (keto at C2, amine at C1)). The resulting α-carbonyl amine is thermodynamically favored over the precursor α-hydroxy imine, thus providing a stable adduct with minimal disruption of the heparin chain. Thus, in this embodiment, the technology provides a heparin chain covalently conjugated at the C1 of the terminal aldose residue of the heparin to an amine containing antithrombin via an amine linkage. If desired, the resulting conjugate may be reduced or labelled by reduction of the C2 carbonyl group with a labelling reagent, such a radiolabel (e.g., NaB³H₄), or conjugated to a second species, such as a fluorescent label.

Heparin molecules can also be classified on the basis of their pentasaccharide content. About one third of heparin contains chains with one copy of the unique pentasaccharide with high affinity for AT, whereas a much smaller proportion (estimated at about 1 wt % of total heparin) includes chains which contain more than one copy of the high affinity pentasaccharide. The remainder (approximately 66%) of the heparin does not contain the pentasaccharide. Thus, the example shown in FIG. 1 that shows the pentasaccharide sequence is by example only, and other molecules of the ATH conjugate of the mixture may not include this sequence, even though the heparin is still attached covalently to the antithrombin protein. Thus, so called “standard heparin” constitutes a mixture of the three species, “low affinity” heparin that lacks a copy of the pentasaccharide, “high affinity” heparin that is enriched for species containing at least one copy of the pentasaccharide, and “very high affinity” heparin that refers to the approximately 1% of molecules that contain more than one copy of the pentasaccharide. These three species can be separated from each other using routine chromatographic methods, such as chromatography over an antithrombin affinity column.

One advantage of forming a conjugate between heparin and a species containing at least one primary amino group (e.g., AT) using the slow glycation process disclosed herein, is the apparent selection for heparin chains having two pentasaccharides. Thus, for example, ATH conjugate prepared by the method of this disclosure appears to be enriched for heparin species containing two pentasaccharides. When standard heparin (containing approximately 1% of two-pentasaccharide heparin) is used as a starting material, usually more than 10% of the resulting ATH conjugate comprises two-pentasaccharide heparin, more often more than about 20%, frequently more than 35%, and often more than about 50% of the ATH conjugate comprises two-pentasaccharide heparin.

This enrichment may account for several useful properties of ATH conjugate. The ATH conjugate of the present technology activates the antithrombin to which it is conjugated, in a stoichiometric fashion, but activates exogenous antithrombin in a catalytic fashion The present technology produces an ATH conjugate with very high specific anti-factor IIa activity.

It will be appreciated that an ATH conjugate of interest (e.g., ATH conjugate) can also be produced by incubating a species containing at least one primary amino group (e.g., AT) with purified very high affinity heparin (i.e., containing two pentasaccharide groups) or a fraction enriched for very high affinity heparin.

In a further aspect of the present disclosure, conjugating antithrombin with heparin outside a body of a subject to form an ATH conjugate can be carried out so that greater than about 60 wt %, greater than about 65 wt %, greater than about 75 wt %, greater than about 85 wt %, greater than about 90 wt %, greater than about 95 wt %, or greater than about 99 wt % based on the starting amount of antithrombin is used in the synthesis. The yield can be increased by various methods. In one example, antithrombin can be conjugated to heparin by the methods described above. Following the conjugation, any unbound antithrombin can be recycled and used in another conjugation reaction with heparin. After each step of incubating antithrombin with heparin, the unbound antithrombin can be recycled and used to make additional ATH conjugate.

In another example, the yield of ATH conjugate can be increased by using an Amadori rearrangement catalyst. Non-limiting examples of catalysts that can increase the rate of Amadori rearrangement include 2-hydroxypyridine, tertiary amine salts, ethyl malonate, phenylacetone, acetic acid, as well as other acids. In a particular example, antithrombin and heparin can be reacted in the presence of 2-hydroxypyridine while being heated in water or very amphiphilic solvents such as formamide. In further examples, antithrombin and heparin can be reacted in the presence of trimethylamine or trimethylamine salts.

The rate of the Amadori rearrangement can also be increased by Amadori rearrangement accelerating solvent systems. Non-limiting examples of solvents include mixtures of water with formamide, dimethylformamide, dioxane, ethanol, dimethylsulfoxide, pyridine, acetic acid, trimethylamine, triethylamine, acetonitrile, and combinations thereof. Heparin and antithrombin can be reacted in these solvent systems to accelerate the Amadori rearrangement to form ATH conjugate.

Increasing the rate of conjugating the heparin aldose to the amine-containing molecule may also involve the use of a linking agent. The linking agent can be a heterobifunctional agent, with a group reactive toward the aldose of heparin at one end and a different group at the other end that can be used for linking either to antithrombin or to a secondary linking agent that can then be linked to AT. In one particular example, the linking agent can contain hydrazine at one end and an amino group at the other end, such as 2-aminoethylhydrazine. This linking agent can be reacted with heparin to form a hydrazone with the aldose aldehyde of the heparin. The product can be dialyzed or diafiltered with membranes that allow heparin chains less than 3,000 or 5,000 Daltons in molecular weight to be removed along with any unreacted linking agent The heparin-hydrazone product can then be reacted with a large excess of a secondary linking agent. The secondary linking agent can be a homobifuntional reagent possessing activated carboxyl groups at each end, such as succinic acid di(N-hydroxysuccinimide) ester (prepared by esterifying succinic acid with N-hydroxysuccinimide using condensing agents such as carbonyldiimidazole or a carbodiimide) so that the amino group on the hydrazine linking agent reacts with just one of the activated carboxyls on the secondary linking agent. The reaction mixture can be dialyzed or diafiltered to remove unreacted secondary linking agent. At this point, the product is heparin modified with the amino-hydrazine linking agent as well as the secondary linking agent. This product can be incubated with antithrombin in buffered H₂O so that the amino group on the antithrombin reacts with the second activated carboxyl group on the secondary linking agent to form an AT-Heparin conjugate, where the antithrombin and heparin are linked by the linking agent and the secondary linking agent.

After forming ATH conjugate, the ATH conjugate can be lyophilized (freeze-dried) for storage. In one embodiment, the ATH conjugate can be prepared in a solution containing only water and then lyophilized. In another embodiment, the ATH conjugate can be prepared in a solution with water and alanine at a concentration of from 0.01-0.09 molar, and then lyophilized. In yet another embodiment, the ATH conjugate can be prepared in a solution containing water and mannitol, and then lyophilized. Each of these methods can be used independently and/or can provide its own advantages. After lyophilization using any of these methods, the ATH conjugate can be reconstituted and retain a significant amount of its activity for inhibiting thrombin compared to its activity prior to lyophilization. Once reconstituted, it can be suitable for delivery, such as via a mist or aerosol as described herein, or by intravenous injection or infusion. In some examples, it may be that the lyophilized powder may be of a particle size that it is suitable for delivery by inhalation as a medicament powder, and can include particles that are small enough to reach the alveoli in some instance. Regardless, in many instances, after lyophilization and reconstitution, the ATH conjugate can retain at least about 50%, at least about 60%, at least about 70%, at least about 80%, at least about 90%, at least about 95%, or at least about 98% of its activity for inhibiting thrombin.

Whether the ATH conjugate has been lyophilized or not, by way of one specific example, the ATH conjugate can be prepared in an aqueous solution containing from 9-11 mg/mL of ATH conjugate with respect to the entire volume of the solution. With a solubility of about 1 wt % in water, forming solutions with more than about 11 mg/mL can lead to aggregation of ATH conjugate. With that stated, there may be instances where some aggregation may be acceptable or even desirable. For example, when delivering to the lungs as droplets, small particulate aggregates of ATH conjugate may be solubilized in vivo over time, and thus, may remain in the lungs for a longer period of time than fully solubilized ATH conjugate. In addition to water, the aqueous carrier may include other additives that may be suitable for administration to humans, including small amounts of organic co-solvent and/or surfactant to assist with droplet size, other proteins, enzymes, solubilizers, adjuvents, buffer, etc. However, in some examples, other additives may be excluded as well and the aqueous carrier may include primarily water, saline, or other pharmaceutically acceptable carrier formulation.

For example, an antiviral solution of ATH conjugate can be delivered as aerosolized droplets from a nebulizer, an inhaler, a nasal mist bottle, or as part of an aerosolized/nebulized single dose from any other similar device, and can include the ATH conjugate in an aqueous carrier as a solution containing water, or water and an additive(s), e.g., simple iso-osmotic salts (such as 0.15 M NaCl) and physiologically acceptable buffers (such as HEPES at pH 7.4), or as described elsewhere herein. In this example, no other protein, solubilizer, adjuvant, or other additives need be present, but could optionally be included without significant deleterious effects on ATH conjugate function. The droplet size that is generated can have an average size (based on droplet number) from about 0.1 μm to about 10 μm, from about 0.3 μm to about 5 μm, from about 0.5 μm to about 2 μm, from about 0.3 μm to about 2. 1μm, from about 1 μm to about 8 μm, or from about 2 μm to about 6 μm, for example.

With these droplet size ranges in mind, when treating for a virus (prophylactically or after viral infection), it may be particularly useful to have a droplet size distribution where an appreciable number of droplets from a size even less than 0.1 μm up to a size that is suitable for the droplets reaching the alveoli of the lungs, as this is a location where virus can be present and thus the subject would benefit from neutralization of the virus within the alveoli. Thus, in one example, the average size of droplets produced by the nebulizer (by droplet count) can be from about 0.1 μm to about 3 μm, from about 0.1 μm to about 2.5 μm, from about 0.3 μm to about 2.1 μm, or from about 0.1 μm to about 1.5 μm. Larger or smaller particles may be present within the droplet size distribution as well, but a distribution of particles having an average droplet size at the lower end of the about 0.1 μm to about 10 μm droplet size range can be particularly useful in causing the ATH conjugate to reach the alveoli at a more appreciable level. That stated, in some examples, when the average droplet size is larger than about 3 μm, there still may be enough droplets in the distribution of a size smaller than about 2.1 μm to reach the alveoli, and if enough of the antiviral solution is aerosolized and delivered, there still may be enough ATH conjugate that reaches the alveoli to treat these deeper lung tissues for the virus. Thus, regardless of the droplet size, delivery of a certain amount of ATH conjugate to the alveoli can be considered one example goal of the treatments and use of the kits/devices described herein. For example, from about 0.05 mg to about 500 mg, from about 0.1 mg to about 400 mg, from about 0.2 mg to about 300 mg, or from about 0.5 mg to about 250 mg of ATH conjugate that is delivered from the aerosolizing device can be delivered to the alveoli to treat the virus. Thus, it is understood that more than these amounts would be delivered in total to the lungs, but these ranges can be used as example target amounts for delivery to the alveoli per single treatment, e.g., period of time on a nebulizer, number of inhalations at one treatment event using an inhaler, etc.

As a note, when delivering antiviral particles of ATH conjugate, these droplet sizes can likewise be relevant to the dry particle sizes that can be delivered using a metered dose, dry medicament inhaler, sometimes referred to as a dry powder inhaler (DPI). This is typically an inhalation-actuated device that delivers the drug in the form of particles from container, such as a capsule or blister punctured prior to use This type of inhaler benefits from an adequate inspiratory flow rate by the user to cause the dose to be delivered to the lungs. It does not typically use pressure or a propellant (as used in liquid nebulizers and/or inhalers). Inhalation by the user is what drives the antiviral particles into the lungs of the user. Again, in some examples, the particle sizes can be as described with respect to the droplets above. In some more specific examples, again, the particle size distribution can be such that an appreciable number of particles can be delivered to the alveoli of the user upon inhalation of the metered dose of antiviral particles of ATH conjugate.

In further reference to the antiviral solution of ATH conjugate that is used to generate the aerosolized droplets, the antiviral solution can be delivered (as droplets) from a volume of fluid from about 0.5 mL to about 1,000 mL, from about 1 mL to about 1,000 mL from about 2 mL to about 500 mL, from about 5 mL to about 500 mL, from about 10 mL to about 500 mL, from about 10 mL to about 250 mL, or from about 25 mL to about 1,000 mL. The amount of ATH conjugate delivered (by weight) during a single dose or single dosing period, e.g., from about 30 seconds to about 2 hours, from about 1 minute to about 90 minutes, from about 5 minutes to about 1 hour, from about 10 minutes to about 45 minutes, or from about 30 minutes to about 2 hours, can be from about 0.1 mg to about 2 g, from about 0.5 mg to about 1 g, from about 1 mg to about 500 mg, from about 5 mg to about 200 mg, or from about 0.5 mg to about 100 mg. In some instances, the treatment duration can be longer than a matter of seconds, e.g., from about 1 minute to about 2 hours, from about 2 minutes to about 2 hours, from about 3 minutes to 2 hours, from 5 minutes to about 2 hours, from about 10 minutes to about 2 hours, or from about 30 minutes to about 2 hours, etc., because unlike treatment of thrombosis where deep alveoli penetration may not be as useful as treating other areas of the lungs, when treating for a viral infection or virus, causing a larger volume of antiviral droplets (and thus content of ATH conjugate) to reach deeper recesses of the alveoli throughout the lungs (to the extent the lungs may allow) can provide an increased therapeutic benefit. Furthermore, by providing droplets having an average droplet size from about 0.1 μm to about 3 μm, from about 0.1 μm to about 2.5 μm, from about 0.3 μm to about 2.1 μm, or from about 0.1 μm to about 1.5 μm, etc., as previously described can also assist with getting more ATH conjugate into the alveoli of the lung tissue. This can be helpful because respiratory viruses, such as coronaviruses, are very small and can be found in these deeper lung tissues. Thus, in one example, a nebulizer or inhaler can be provided for use that includes a volume of antiviral solution (and ATH conjugate content) suitable for longer duration treatments and/or that can deliver a higher volume of smaller droplet sizes to the lungs, thus allowing more ATH conjugate to reach the alveoli.

ATH conjugate can alternatively be delivered intravenously by infusion or injection as a single dose or part of a dosing regimen in an aqueous carrier in the form of a solution containing simple iso-osmotic salts (such as 0.15 M NaCl) and physiologically acceptable buffers (such as HEPES at pH 7.4). In this example, no other protein, solubilizer, adjuvant, or other additives need be present, but could optionally be included without significant deleterious effects on ATH conjugate function. In one example, ATH conjugate can be administered intravenously at a dosage ranging from 1 unit (in terms of anti-factor Xa activity) per kilogram (unit/kg) up to 1000 units/kg, with a typical dose being about 50 units/kg to about 300 units/kg. If ATH conjugate has an anti-factor Xa activity of 130 units per milligram (units/mg), in terms of the antithrombin component of ATH conjugate, then ATH conjugate dosages can range from about 0.008 mg up to about 8 mg, with a typical dose being about 0.3 mg to about 5 mg. Given ATH conjugate's longer intravenous half-life than heparin, ATH conjugate may not need to be administered frequently, but it is safe to do so because ATH conjugate has little risk of unwanted bleeding. Thus, ATH conjugate could be administered twice per day, once per day, once every other day, twice per week, or even once per week until the viral infection is under control or the viral load is undetectable in the subject

With these more specific examples above in mind, in further detail, an acceptable daily dose of about 0.001 mg to about 50 mg per kilogram body weight of the recipient per day can be delivered. In some examples, about 0.05 mg to about 25 mg per kilogram body weight per day, or about 0.01 to about 10 mg per kilogram body weight per day can be delivered, depending on the severity of the viral symptoms being treated, whether the treatment is for purposes of prophylaxis, or depending on the route of administration. For example, if delivering as an aerosol directly to lung tissue to treat a lung infection, it may be appropriate to deliver less ATH conjugate than may be used if delivering systemically by some other delivery route. In still other examples, it may be appropriate to deliver the antiviral solution of ATH conjugate during an invasive medical procedure to ameliorate the risk of viral infection and also lower risk of thromboembolic complications, as in some instances, there may be increased risk of viral infections in hospitals or medical surgical centers.

It is also understood that terms used herein will take on the ordinary meaning in the relevant technical field unless specified otherwise. In some instances, there are terms defined more specifically throughout the specification or included below, and thus, these more specifically defined terms have a meaning as defined, unless the context clearly dictates otherwise.

The singular forms “a,” “an,” and “the” include plural references unless the context clearly dictates otherwise. Thus, for example, reference to “an organic cosolvent” includes reference to one or more of such components, or “a mixing step” refers to one or more of such steps.

As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Furthermore, the term “about” expressly includes the exact range parameters set forth in the range that uses “about.” Thus, about 1 to about 5 may allow for minimal flexibility of range parameters, but also directly supports the exact range of from 1 to 5.

“Heparin” is a sulfonated polysaccharide which is typically an alternating sequence of hexuronic acid and 2-amino-2-deoxy-D-glucose. Anticoagulation using GAGs (such as heparin and dermatan sulfate) proceeds via their catalysis of inhibition of coagulant enzymes (a significant one being thrombin) by serine protease inhibitors (serpins) such as antithrombin III (referred to herein as simply “antithrombin” or “AT”) and heparin cofactor II (HCII). Binding of the serpins by the catalysts occurs for their action and occurs through specific sequences along the linear carbohydrate chain of the glycosaminoglycan (GAG). Heparin, for example, acts by binding to antithrombin via a pentasaccharide sequence, thus potentiating inhibition of a variety of coagulant enzymes (in the case of thrombin, heparin also binds to the enzyme). Heparin can also potentiate inhibition of thrombin by binding to the serpin HCII.

In accordance with the present disclosure, an “antithrombin-heparin conjugate” or “ATH conjugate” refers to antithrombin covalently bonded to heparin, and thus, can be more permanent that natural complexes of antithrombin and heparin. As heparin can bind to other molecules in vivo or be lost from its pentasaccharide attraction site due to any of a number of mechanisms or interactions, to keep heparin more permanently associated with the antithrombin, a covalent bond can be present, which is a separate point of attachment as opposed to the non-covalent pentasaccharide sequence attachment. The heparin of ATH conjugate can retain its biological activity, and in some instances, may be strengthened, e.g., binding power to viruses, and it can be more stable and effective for use in treating viruses and thrombosis that may be associated with viral infection.

As used herein, “serpin” refers to a serine protease inhibitor and is exemplified by species such as antithrombin and heparin cofactor II.

As used herein, “amine” refers to the type of amine that is chemically relevant in the context of its use, and may include primary amines, secondary amines, tertiary amines, and/or quaternary amines. The term amino, on the other hand, refers to primary amines, e.g., NH or NH₂. The term “imine” refers to the group C═N and salts thereof.

As used herein, the terms “treatment” or “treating” of a condition and/or a disease in a subject, such as a human, includes preventing the condition or disease, that is, avoiding any clinical symptoms of the disease; inhibiting the condition or disease, that is, arresting the development or progression of clinical symptoms; and/or relieving or ameliorating the condition or disease, that is, causing the regression of clinical symptoms and/or retreat of infection associated therewith. Treatment also includes use of the compositions of the present disclosure associated with a medical procedure with administration before, during, or after an infection, or in some cases, an infection or to prevent an infection associated with a medical procedure.

The term “single treatment” refers to the delivery of the ATH conjugate to a subject in a specific time or time period For example, with a nebulizer, a single treatment would include the time period breathing (inhaling droplets and exhaling) at a single sitting, e.g., 30 seconds to 2 hours. There may be multiple “single treatments” during the course of a day or the course of a few days or the course of a week or month, for example. A single treatment using a liquid droplet inhaler or a dry medicament inhaler may be a single inhaled dose or multiple inhaled dosages that are given at during a single narrow period of time, e.g., one inhalation, 5 inhalations within 5 minutes, etc. A single treatment for an intravenous infusion would include the time period where the dosage to be administered is received by the subject

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and 200, but also to include individual sizes such as 2, 3, 4, and sub-ranges such as 10 to 50, 20 to 100, etc.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

### EXAMPLES

The following examples illustrate embodiments of the disclosure. However, it is to be understood that the following are only exemplary or illustrative of the application of the principles of the present technology. Numerous modifications and alternative compositions, methods and/or systems may be devised without departing from the scope of the present disclosure. The appended claims are intended to cover such modifications and arrangements. Thus, while the present disclosure has been described above with particularity, the following examples provide further detail in connection with what are presently deemed to be practical embodiments of the disclosure.

### Example 1—Preparation of ATH Conjugates

Heparin is reacted with antithrombin as described in U.S. Pat. No. 7,045,585 to form ATH conjugates for use in accordance with the present disclosure. However, in some examples, it may be desirable to remove low molecular weight heparin to a large extent prior to forming the ATH conjugates. In such instances, the heparin is separated from low molecular weight heparin and then reacted with antithrombin.

Heparin can be processed to remove the low molecular weight heparin by filtration, for example. In this instance, about 0.5 mL of 10,000 I.U./mL of heparin is filtered in a 49 cm by 1 cm chromatography column and eluted with 1 M NaCL and multiple 1.27 g fractions are collected. Using this protocol, absorbency of the samples (A215) versus water was used to verify samples substantial removal of low molecular weight heparin. Some of the samples had weight average molecular weights of about 18,000 Daltons with low molecular weight heparin substantially removed.

In other examples, high molecular weight heparin samples are prepared that are substantially devoid of low molecular weight heparin are pressure-dialyzed to a concentration of 13.87 mg/mL and then further dialyzed against 0.02 M phosphate 0.15 M NaCl pH 7.3 at 4° C. The samples in either scenario can be freeze-dried or stored at −60° C.

To prepare the ATH conjugate, about 19.12 mg of a heparin sample is dissolved in 1 mL of 0.3 M disodium phosphate 1 M NaCl pH 9.5, which had been filtered through a sterile 0.2 micron pore size acrodisc. The resultant solution is placed in a 12 mm by 75 mm plastic test tube and 72 μL of human antithrombin is added with mixing. The tube is closed with a plastic cap and sealed around the outside of the cap with parafilm. The tube and contents are then heated in a water bath at 37° C. for 14 days. After the 14 day incubation, the mixture of heparin and antithrombin forms are gel filtered on a 48.5 cm by 1 cm column of Sephadex® G-200 with 1 M NaCl and 20-drop fractions were collected.

### Example 2—Preparation and Delivery of Antiviral Solution of ATH Conjugate to the Lungs

ATH conjugate is obtained that includes antithrombin covalently attached to heparin at a first end of the heparin. In this example, the average monosaccharide length of the heparin attached to the antithrombin is about 300, with the heparin chain having a weight of about 77,100 to about 86,850 Daltons, e.g., use of disaccharide with an average of about 2-3 sulfonate groups, though closer to 3 is more common. To a 100 mL aqueous carrier including primarily water with additives including about 2 wt % buffer, the ATH conjugate was dissolved into the aqueous carrier at 0.1 wt %. The 0.1 wt % antiviral solution of ATH conjugate is loaded in a nebulizer cup, and the solution is nebulized, e.g., jet nebulized, to form droplets having an average droplet size of about 1-2 μm for inhalation into the lungs of a subject through a mouthpiece or facemask. In this example, the droplet size distribution may also include a portion of the droplets having a droplet size less than 1 μm, which can be particularly useful for reaching the alveoli of the lungs. It is estimated that the 100 mL of the 0.1 wt % ATH conjugate antiviral solution can be delivered into the branches and alveoli of the lungs in about 15 minutes of use, with about 100 mg of the ATH conjugate being deposited throughout the lungs for treatment of a virus.

### Example 3—Preparation and Delivery of Antiviral Solution of ATH Conjugate to the Lungs

ATH conjugate is obtained that includes antithrombin covalently attached to heparin at a first end of the heparin. In this example, the average monosaccharide length of the heparin attached to the antithrombin is about 100, with the heparin chain having a weight of about 28,950 Daltons. To a 100 mL aqueous carrier including primarily water with additives including about 2 wt % buffer, the ATH conjugate was dissolved into the aqueous carrier at 0.5 wt %. The 0.5 wt % antiviral solution of ATH conjugate is loaded in a pump inhaler so that droplets of the antiviral solution are formed and delivered through a mouthpiece each time the inhaler is pumped. It is estimated that each time the inhaler is pumped, about 100 mL of the 0.5 wt % ATH conjugate antiviral solution can be delivered into the lungs with inhalation by the subject for treatment of a virus.

### Example 4—Preparation and Delivery of Antiviral Solution of ATH Conjugate to Systemic Circulation

ATH conjugate is obtained that includes antithrombin covalently attached to heparin at a first end of the heparin. In this example, the average monosaccharide length of the heparin attached to the antithrombin is about 200, with the heparin chain having a weight of about 57,900 Daltons. To a 250 mL aqueous carrier including primarily saline with 0.15 M NaCl and HEPES (or other physiologically acceptable buffer) having a pH of about 7.4 is added about 0.1 wt % of the ATH conjugate. The 0.1 wt % antiviral solution of ATH conjugate is loaded in an intravenous drip system and delivered directly into the vein of a subject by infusion for a period of time of about 60 minutes. The ATH conjugate is thus delivered into systemic circulation for treatment of a virus.

It is to be understood that the above-referenced arrangements are illustrative of the application for the principles of the present disclosure. Thus, while the present technology has been described above in connection with the exemplary embodiments, it will be apparent to those of ordinary skill in the art that numerous modifications and alternative arrangements can be made without departing from the principles and concepts of the disclosure as set forth in the claims.

What is claimed is:

1. A method of treating respiratory infection, comprising delivering aerosolized droplets of an antiviral solution to respiratory tissue of a subject, the antiviral solution including an aqueous carrier and an ATH conjugate that is solubilized in the aqueous carrier, wherein the ATH conjugate includes antithrombin covalently bonded to heparin, the heparin including a polysaccharide chain having from about 15 to about 1,500 monosaccharide units.

2. The method of claim 1, wherein the aerosolized droplets are delivered to lung tissue using a nebulizer.

3. The method of claim 1, wherein the aerosolized droplets are delivered to lung using an inhaler.

4. The method of claim 1, wherein the aerosolized droplets are delivered to nasal tissue, sinus tissue, epiglottis tissue, or a combination thereof using a sprayer or sinus rinse device.

5. The method of claim 1, wherein delivering the aerosolized droplets occurs by inhalation while the subject is breathing, and wherein delivering occurs for a period of time from about 30 seconds to about 2 hours.

6. The method of claim 1, wherein delivering the aerosolized droplets occurs by inhalation of from 1 inhalation dose to about 10 inhalation doses.

7. The method of claim 1, wherein the ATH conjugate is delivered to lung tissue and remains at the lung tissue of the subject and does not substantially enter the bloodstream for at least 1 day.

8. The method of claim 1, wherein the ATH conjugate is present as a mixture of ATH conjugates in the antiviral solution, wherein the mixture of ATH conjugates have an average polysaccharide chain length from about 10 to about 1,200 monosaccharide units.

9. The method of claim 8, wherein the average polysaccharide chain length is from about 18 to about 1,000 monosaccharide units.

10. The method of claim 8, wherein the average polysaccharide chain length is from about 50 to about 500 monosaccharide units.

11. The method of claim 1, wherein from about 98% to 100% of the heparin chains of the ATH conjugate have an average molecular weight of at least about 3,000 Daltons.

12. The method of claim 1, wherein from about 98% to 100% of the heparin chains of the ATH conjugate have an average molecular weight of at least about 5,000 Daltons.

13. The method of claim 1, wherein from about 98% to 100% of the heparin chains of the ATH conjugate have an average molecular weight of at least about 7,500 Daltons.

14. The method of claim 1, wherein the antithrombin is covalently bonded to the polysaccharide chain at a monosaccharide positioned at a first end of a polysaccharide chain.

15. The method of claim 1, wherein the ATH conjugate includes a polysaccharide chain having a chain length, a configuration relative to the antithrombin, or both that inhibits viral- or vaccine-induced thrombosis at the respiratory tissue.

16. The method of claim 1, wherein the polysaccharide chain includes a pentasaccharide sequence that interacts with the antithrombin, wherein a first side of the pentasaccharide sequence includes a polysaccharide residue having a monosaccharide chain length from about 2 to about 15 and further includes a monosaccharide unit that is covalently bonded to the antithrombin, and wherein a second side of the pentasaccharide sequence includes a free polysaccharide chain having a monosaccharide chain length from about 10 to about 1,500 monosaccharides.

17. The method of claim 1, wherein the ATH conjugate is present in the aqueous carrier at from about 0.005 wt % to about 2 wt %.

18. The method of claim 1, wherein the ATH conjugate is fully solubilized in the aqueous carrier and is present in the aqueous carrier at from about 0.01 wt % to about 1 wt %.

19. The method of claim 1, wherein the aerosolized droplets have an average droplet size from about 1 μm to about 200 μm based on droplet number.

20. The method of claim 1, wherein the aerosolized droplets have an average droplet size from about 0.1 μm to about 10 μm based on droplet number.

21. The method of claim 1, wherein the aerosolized droplets have an average droplet size from about 0.3 μm to about 2.1 μm based on droplet number.

22. The method of claim 1, wherein the antiviral solution is delivered to the subject as the aerosolized droplets at volume from about 0.5 mL to about 1,000 mL.

23. The method of claim 1, wherein the antiviral solution is delivered to the subject as the aerosolized droplets at volume from about 10 ml to about 500 ml.

24. The method of clam 1, wherein from about 0.01 mg to about 2 g of the ATH conjugate is delivered to respiratory tissue during a single treatment.

25. The method of clam 1, wherein from about 0.1 mg to about 2 g of the ATH conjugate is delivered to lung tissue during a single treatment.

26. The method of clam 1, wherein from about 5 mg to about 200 mg of the ATH conjugate is delivered to lung tissue during a single treatment.

27. The method of claim 1, wherein from about 0.05 mg to about 500 mg of the ATH conjugate is delivered to alveoli of the lung tissue during a single treatment.

28. The method of claim 1, wherein after a single treatment, detectable levels of the ATH conjugate remain deposited at the lung tissue for at least 7 days.

29. The method of claim 1, wherein after a single treatment, detectable levels of the ATH conjugate remain deposited at the lung tissue for at least 4 days.

30. The method of claim 1, wherein the method of treating respiratory infection is by preventing infection from a virus at the lung tissue.

31. The method of claim 1, wherein the method of treating respiratory infection is by reducing or eliminating a viral load of the virus at the lung tissue.

32. The method of claim 1, wherein the respiratory infection is a viral infection from a coronavirus.

33. The method of claim 32, wherein the coronavirus is SARS-COV-2, SARS-COV-1, or a mutation or derivative thereof.

34. The method of claim 32, wherein an electronegative moiety on the heparin of the ATH conjugate binds to a spike protein of the coronavirus.

35. The method of claim 1, wherein the respiratory infection is a viral infection from an influenza virus, a respiratory syncytial virus (RSV), a parainfluenza virus, a human metapneumovirus (HMPV), a rhinovirus, an adenovirus, or a human bocavirus (HBoV).

36. The method of claim 1, wherein the respiratory infection is a vaccine-induced infection.

37. A respiratory tissue treatment kit or device, comprising:
an antiviral solution including an aqueous carrier and an ATH conjugate that is solubilized in the aqueous carrier, wherein the ATH conjugate includes antithrombin covalently bonded to heparin, the heparin including a polysaccharide chain having from about 15 to about 1,500 monosaccharide units; and
an aerosolizing device loaded or loadable with the antiviral solution to form aerosolized droplets of the antiviral solution for delivery to respiratory tissue of a subject.

38. The respiratory tissue treatment kit of claim 37, wherein the antiviral solution is loaded or loadable in the aerosolizing device at a sufficient volume to deliver the aerosolized droplets during a single treatment for a period of time from about 30 seconds to about 2 hours.

39. The respiratory tissue treatment kit of claim 38, the antiviral solution is delivered to the subject by inhalation, and wherein the sufficient volume delivers during the period of time delivers about 0.05 mg to about 500 gm of the ATH conjugate to the alveoli of lung tissue during the single treatment.

40. The respiratory tissue treatment kit of claim 38, wherein the antiviral solution is loaded or loadable in the aerosolizing device at a sufficient volume to deliver the aerosolized droplets using one or multiple single inhalation dosages to deliver about 0.05 mg to about 500 mg of the ATH conjugate to the alveoli of the lungs during a single treatment.

41. The respiratory tissue treatment kit of claim 37, wherein the aerosolized droplets are generated by a nebulizer that includes a mouthpiece or facemask for inhalation of the aerosolized droplets.

42. The respiratory tissue treatment kit of claim 37, wherein the aerosolized droplets are generated by an inhaler that includes a mouthpiece or facemask for inhalation of the aerosolized droplets.

43. The respiratory tissue treatment kit of claim 37, wherein the aerosolized droplets are generated by a sprayer or sinus rinse device that includes a nozzle for application to nasal tissue, sinus tissue, epiglottis, or a combination thereof.

44. The respiratory tissue treatment kit of claim 37, wherein the ATH conjugate is configured so that when deposited at the respiratory tissue of the subject, the ATH conjugate does not substantially enter the bloodstream.

45. The respiratory tissue treatment kit of claim 37, wherein the ATH conjugate is present as a mixture of ATH conjugates in the antiviral solution, wherein the mixture of ATH conjugates have an average polysaccharide chain length from about 10 to about 1,200 monosaccharide units.

46. The respiratory tissue treatment kit of claim 44, wherein the average polysaccharide chain length is from about 18 to about 1,000 monosaccharide units.

47. The respiratory tissue treatment kit of claim 37, wherein the average polysaccharide chain length is from about 50 to about 500 monosaccharide units.

48. The respiratory tissue treatment kit of claim 37, wherein from about 98% to 100% of the heparin chains of the ATH conjugate have an average molecular weight of at least about 3,000 Daltons.

49. The respiratory tissue treatment kit of claim 37, wherein from about 98% to 100% of the heparin chains of the ATH conjugate have an average molecular weight of at least about 5,000 Daltons.

50. The respiratory tissue treatment kit of claim 37, wherein from about 98% to 100% of the heparin chains of the ATH conjugate have an average molecular weight of at least about 7,500 Daltons.

51. The respiratory tissue treatment kit of claim 37, wherein the antithrombin is covalently bonded to the polysaccharide chain at a monosaccharide positioned at a first end of a polysaccharide chain.

52. The respiratory tissue treatment kit of claim 37, wherein the ATH conjugate includes a polysaccharide chain having a chain length, a configuration relative to the antithrombin, or both that inhibits viral- or vaccine-induced thrombosis at the respiratory tissue.

53. The respiratory tissue treatment kit of claim 37, wherein the polysaccharide chain includes pentasaccharide sequence that interacts with the antithrombin, wherein a first side of the pentasaccharide sequence includes a polysaccharide residue having a monosaccharide chain length from about 2 to about 15 and further includes a monosaccharide unit that is covalently bonded to the antithrombin, and wherein a second side of the pentasaccharide sequence includes a free polysaccharide chain having a monosaccharide chain length from about 10 to about 1,500 monosaccharides.

54. The respiratory tissue treatment kit of claim 37, wherein the ATH conjugate is present in the aqueous carrier at from about 0.005 wt % to about 2 wt %.

55. The respiratory tissue treatment kit of claim 37, wherein the ATH conjugate is fully solubilized in the aqueous carrier and is present in the aqueous carrier at from about 0.01 wt % to about 1 wt %.

56. The respiratory tissue treatment kit of claim 37, wherein the aerosolizing device is configured to generate the aerosolized droplets at an average droplet size from about 1 μm to about 200 μm based on droplet number.

57. The respiratory tissue treatment kit of claim 37, wherein the aerosolizing device is configured to generate the aerosolized droplets at an average droplet size from about 0.1 μm to about 10 μm based on droplet number.

58. The respiratory tissue treatment kit of claim 37, wherein the aerosolizing device is configured to generate the aerosolized droplets at an average droplet size from about 0.3 μm to about 3 μm based on droplet number.

59. The respiratory tissue treatment kit of claim 37, wherein a single dose of the antiviral solution for delivery to the subject is from about 0.5 mL to about 1,000 ml.

60. The respiratory tissue treatment kit of claim 37, wherein a single dose of the antiviral solution for delivery to the subject is from about 10 mL to about 500 mL.

61. The respiratory tissue treatment kit of claim 37, wherein a single dose of the antiviral solution for delivery to the subject delivers from about 0.1 mg to about 2 g of the ATH conjugate.

62. The respiratory tissue treatment kit of claim 37, wherein a single dose of the antiviral solution for delivery to the subject delivers from about 5 mg to about 200 mg of the ATH conjugate.

63. A respiratory tissue treatment kit or device, comprising:
antiviral particles including ATH conjugate, wherein the ATH conjugate includes antithrombin covalently bonded to heparin, the heparin including a polysaccharide chain having from about 15 to about 1,500 monosaccharide units; and
a dry medicament inhaler loaded or loadable with the antiviral providing a metered dose or multiple metered dosages of the antiviral particles for inhalation.

64. The respiratory tissue treatment kit or device of claim 63, wherein the antiviral particles have an average particle size from about 0.1 μm to about 10 μm based on particle number.

65. The respiratory tissue treatment kit or device of claim 63, wherein the antiviral particles are lyophilized particles of an antiviral solution containing the ATH conjugate.

66. The respiratory tissue treatment kit or device of claim 63, wherein from about 98% to 100% of the heparin chains of the ATH conjugate have an average molecular weight of at least about 3,000 Daltons.

67. A method of treating infection, comprising delivering an antiviral composition to a subject, wherein the antiviral composition is in the form of an antiviral solution or antiviral particles, the antiviral composition including an ATH conjugate including antithrombin covalently bonded to heparin, the heparin including a polysaccharide chain having from about 15 to about 1,500 monosaccharide units.

68. The method of claim 67, wherein the antiviral composition is the antiviral solution, and the antiviral solution is delivered by an administration route selected from parenteral, intravenous, subcutaneous, intramuscular, oral, dermal, ocular, or mucosal, or inhalation.

69. The method of claim 68, wherein the antiviral solution includes fully solubilized ATH conjugate in an aqueous carrier suitable for intravenous infusion or subcutaneous injection, and wherein delivery is by intravenous infusion or subcutaneous injection.

70. The method of claim 69, wherein antiviral composition is the antiviral solution, and the ATH conjugate is present in the aqueous carrier at from about 0.005 wt % to about 2 wt %.

71. The method of clam 69, wherein from about 0.1 mg to about 2 g of the ATH conjugate is delivered to the subject during a single treatment.

72. The method of claim 69, wherein a single dose of the antiviral solution for delivery to the subject is from about 0.5 mL to about 1,000 mL.

73. The method of claim 67, wherein the antiviral composition are the antiviral particles, and the antiviral particles are delivered by an administration route selected from oral, mucosal, or inhalation.

74. The method of claim 67, wherein the antiviral composition is in the form of solid particles or powder, semi-solid particles, a solubilized liquid, a liquid dispersion, a compressed tablet, a suppository, a liquid-containing soft-gel capsule, a particle-containing capsule, or an emulsion.

75. The method of claim 67, wherein the ATH conjugate is present as a mixture of ATH conjugates in the antiviral composition, wherein the mixture of ATH conjugates have an average polysaccharide chain length from about 10 to about 1,200 monosaccharide units.

76. The method of claim 67, wherein the average polysaccharide chain length is from about 50 to about 1,000 monosaccharide units.

77. The method of claim 67, wherein from about 98% to 100% of the heparin chains of the ATH conjugate have an average molecular weight of at least about 3,000 Daltons.

78. The method of claim 67, wherein the antithrombin is covalently bonded to the polysaccharide chain at a monosaccharide positioned at a first end of a polysaccharide chain.

79. The method of claim 67, wherein the polysaccharide chain includes pentasaccharide sequence that interacts with the antithrombin, wherein a first side of the pentasaccharide sequence includes a polysaccharide residue having a monosaccharide chain length from about 2 to about 15 and further includes a monosaccharide unit that is covalently bonded to the antithrombin, and wherein a second side of the pentasaccharide sequence includes a free polysaccharide chain having a monosaccharide chain length from about 10 to about 1,000 monosaccharides.

80. The method of claim 67, wherein the infection is a viral infection from a coronavirus.

81. The method of claim 80, wherein the coronavirus is SARS-COV-2, SARS-COV-1, or a mutation or derivative thereof.

82. The method of claim 80, wherein an electronegative moiety of the heparin binds to a spike protein of the coronavirus.

83. The method of claim 67, wherein the infection is a viral infection from an influenza virus, a respiratory syncytial virus (RSV), a parainfluenza virus, a human metapneumovirus (HMPV), a rhinovirus, an adenovirus, or a human bocavirus (HBoV).

84. The method of claim 67, wherein the infection is a vaccine-induced infection.
